(19) United States
(12) Patent Application Publication
(10) Pub. No.: US 2026/0191915 A1
(43) Pub. Date: Jul. 9, 2026

(54) EcNc ENGINEERED STRAIN CONSTITUTIVELY EXPRESSING MLH PROTEIN AND USE THEREOF IN TREATMENT OF ENTERITIS

(71) Applicant: Angelo (Shenzhen) Biotechnology Co. Ltd, Shenzhen (CN)

(72) Inventors: Zuoming NIE, Shenzhen (CN); Qi LIN, Shenzhen (CN); Zhuo JIANG, Shenzhen (CN); Zhan LUO, Shenzhen (CN); Dan WANG, Shenzhen (CN); Zhengbing LV, Shenzhen (CN)

(73) Assignee: Angelo (Shenzhen) Biotechnology Co. Ltd, Shenzhen (CN)

(21) Appl. No.: 19/439,937

(22) Filed: Jan. 5, 2026

(30) Foreign Application Priority Data
Jan. 7, 2025 (CN) 202510022206.2

(51) Int. Cl. A61K 35/74; A61P 1/04; C12N 1/20; C12N 9/02; C12N 9/08; C12N 15/62; C12N 15/75; C12R 1/19

(52) U.S. Cl. CPC A61K 35/74; A61P 1/04; C12N 1/20; C12N 9/0065; C12N 9/0089; C12N 15/62; C12N 15/75; C07K 2319/00; C12R 2001/19; C12Y 111/01006; C12Y 115/01001

(57) ABSTRACT

An EcNc engineered strain constitutively expressing an MLH protein and the use thereof in the treatment of enteritis, belonging to the field of biopharmaceutical technology. The present disclosure provides an EcNc engineered strain constitutively expressing an MLH protein, where Escherichia coli Nissle 1917 (EcN) is used as a chassis cell, from which two cryptic plasmids are removed, followed by introducing the modified endogenous cryptic plasmids pMUT1 and/or pMUT2; the modification refers to inserting a gene encoding the MLH fusion protein formed by linking SOD and CAT via a flexible linker into pMUT1 and/or pMUT2. In the present disclosure, an engineered strain constitutively expressing a fusion protein is constructed, where the engineered strain can express a fusion protein with SOD and CAT dual enzyme activities without antibiotic stress and IPTG induction. The constructed engineered strain can be used as a live biotherapeutic microorganism for the treatment of UC diseases.

### CROSS REFERENCE TO RELATED APPLICATION

This patent application claims the benefit and priority of Chinese Patent Application No. 202510022206.2 filed with the China National Intellectual Property Administration on Jan. 7, 2025, the disclosure of which is incorporated by reference herein in its entirety as part of the present application.

### REFERENCE TO SEQUENCE LISTING

A computer readable XML file entitled “GWP20250902237”, that was created on Dec. 5, 2025, with a file size of about 21,087 bytes, contains the sequence listing for this application, has been filed with this application, and is hereby incorporated by reference in its entirety.

### TECHNICAL FIELD

The present disclosure belongs to the field of biopharmaceutical technology, and specifically relates to an EcNc engineered strain constitutively expressing an MLH protein and the use thereof in the treatment of enteritis.

### BACKGROUND

Ulcerative colitis (UC) is a chronic inflammatory bowel disease (IBD) with an etiology that has not yet been fully elucidated. The main clinical symptoms of UC include bloody diarrhea, abdominal pain, bowel urgency and tenesmus, and may be accompanied by extraintestinal manifestations, such as systemic symptoms like anemia, fever, and weight loss. As such, UC is a disease that severely impacts human health. Traditional chemical agents for UC lead to incomplete treatment and unpredictable disease recurrence, which causes harm to the physical and mental health of patients. Chemical drugs can only alleviate symptoms rather than fundamentally cure UC, and often cause significant adverse effects on other organs in the body. Moreover, gastrointestinal drugs currently available on the market have a relatively one-sided and limited effect in treating chronic colitis, that is, they can either only repair the mucosa without eliminating pathogenic bacteria, or only kill pathogenic bacteria without providing mucosal repair.

In recent years, emerging therapeutic strategies such as biologics and fecal microbiological transplantation (FMT) have shown significant value in the treatment of UC. Biologics have shown significant efficacy, particularly in alleviating moderate-to-severe cases of ulcerative colitis (UC). However, there is currently no live biotherapeutic product with significant effects available for the treatment of UC.

### SUMMARY

The present disclosure provides an EcNc engineered strain constitutively expressing an MLH protein and the use thereof in the treatment of enteritis. Specifically, an engineered strain EcNc-MLH constitutively expressing a fusion protein RtSOD-CAT (MLH) has been developed, which can be used as a live biotherapeutic product (LBP) for the prevention and treatment of enteritis.

The present disclosure provides an EcNc engineered strain constitutively expressing an MLH protein, where Escherichia coli Nissle 1917 (EcN) is used as a chassis cell, and contains modified endogenous cryptic plasmids pMUT1 and/or pMUT2;
the modification includes inserting a gene encoding an MLH protein into the endogenous cryptic plasmids pMUT1 and/or pMUT2, where the MLH protein is a fusion protein formed by linking SOD and CAT via a flexible linker.

In one embodiment of the present disclosure, the SOD includes RtSOD with the GenBank Accession No. WP_012843426.1; the CAT includes FeCAT with the GenBank Accession No. WP_012845258.1.

The present disclosure provides a construction method for the above EcNc engineered strain, which method includes steps of: (1) inserting a gene encoding SOD into endogenous cryptic plasmids pMUT1 and/or pMUT2, and constructing linearized pMUT1-SOD and/or pMUT2-SOD plasmids by PCR method;
(2) inserting a gene encoding CAT into a prokaryotic expression vector, and constructing a linearized linker-CAT gene fragment by PCR method;
(3) ligating the linearized pMUT1-SOD and/or pMUT2-SOD plasmids of step (1) separately with the linearized linker-CAT gene fragment of step (2), to construct recombinant vectors pMUT1-MLH and/or pMUT2-MLH; and
(4) transforming the recombinant vectors pMUT1-MLH and/or pMUT2-MLH of step (3) separately into Escherichia coli Nissle 1917 with the two endogenous cryptic plasmids pMUT1 and pMUT2 removed, to construct the EcNc engineered strain.

In one embodiment of the present disclosure, primers used in the PCR method of step (1) include pMRtSOD-X-F and pMRtSOD-X-R, where the nucleotide sequence of the pMRtSOD-X-F is set forth in SEQ ID NO: 1, and the nucleotide sequence of the pMRtSOD-X-R is set forth in SEQ ID NO: 2.

In one embodiment of the present disclosure, primers used in the PCR method of step (2) include L-FeCAT-F and L-FeCAT-R, where the nucleotide sequence of the L-FeCAT-F is set forth in SEQ ID NO: 3, and the nucleotide sequence of the L-FeCAT-R is set forth in SEQ ID NO: 4.

The present disclosure further provides the use of the above EcNc engineered strain or an EcNc engineered strain constructed using the above construction method in the preparation of an antioxidant formulation.

The present disclosure further provides the use of the above EcNc engineered strain or an EcNc engineered strain constructed using the above construction method in the preparation of a drug for preventing and/or treating enteritis.

In one embodiment of the present disclosure, types of enteritis include ulcerative colitis.

In one embodiment of the present disclosure, types of the drug for preventing and/or treating enteritis include a live biotherapeutic product.

The present disclosure further provides a drug for preventing and/or treating enteritis, where the drug contains an active ingredient including the above EcNc engineered strain or an EcNc engineered strain constructed using the above construction method, and further comprises a pharmaceutically acceptable excipient.

Beneficial effects: The present disclosure provides an EcNc engineered strain constitutively expressing an MLH protein, where Escherichia coli Nissle 1917 (EcN) is used as a chassis cell, from which two cryptic plasmids are removed, followed by introducing the modified endogenous cryptic plasmids pMUT1 and/or pMUT2; the modification refers to inserting a gene of an MLH fusion protein formed by linking SOD and CAT via a flexible linker into pMUT1 and/or pMUT2. In the present disclosure, an engineered strain EcNc-MLH constitutively expressing a fusion protein RtSOD-CAT (MLH) is constructed, which can express a dual-enzyme fusion protein of SOD and CAT without antibiotic pressure or IPTG induction. Moreover, the engineered strain EcNc-MLH has SOD and CAT enzyme activities, and remains relatively stable in an environment at pH 3-11 and in artificial intestinal fluid, thereby further enhancing the application value of the EcN engineered strain.

The engineered strain EcNc-MLH constructed in the present disclosure can significantly reduce dextran sulphate sodium (DSS)-induced intestinal inflammation in mice and promote intestinal mucosal healing in UC mice. Biochemical analysis shows that the contents of SOD and CAT in intestinal tissues are significantly increased, the content of malondialdehyde (MDA) is significantly reduced, and the mRNA expression levels of the inflammatory factors IL-6 and IL-1β are also decreased. The engineered strain EcNc-MLH of the present disclosure can be used as an engineered microorganism for the treatment of UC diseases, for example, as a live biotherapeutic product (LBP) for the prevention and treatment of enteritis, and thus has good application prospects.

### BRIEF DESCRIPTION OF THE DRAWINGS

FIGS. 1A-1B shows PCR electrophoresis images for the construction of plasmid pMUT1-MLH, in which A: linker-FeCAT target fragment; and B: linearized pMUT1-RtSOD vector;

FIGS. 2A-2B shows the construction of the EcNc-MLH engineered strain expressing the MLH fusion protein and the results of protein expression identification, in which FIG. 2A: schematic diagram of recombinant cryptic plasmids pMUT1-MLH and pMUT2-MLH; and FIG. 2B: SDS-PAGE identification of the constitutive expression of the recombinant MLH fusion protein in the EcN engineered strain, where band 1 indicates no IPTG induction; band 2 indicates induction with IPTG;

FIGS. 3A-3D shows the results of the MLH stability test, in which FIG. 3A: high-temperature resistance test of SOD and CAT enzymes of MLH; FIG. 3B: acid-base resistance test of SOD and CAT enzymes in MLH; FIG. 3C: artificial gastric juice resistance test of SOD and CAT enzymes in MLH; and FIG. 3D: artificial intestinal juice resistance test of SOD and CAT enzymes in MLH;

FIGS. 4A-4G shows the results of therapeutic evaluation of the EcNc-MLH engineered strain on ulcerative colitis (UC) in mice, in which FIG. 4A: changes in body weight of mice; FIG. 4B: changes in DAI of mice; FIG. 4C: macroscopic changes in colon length of mice; FIG. 4D: statistical analysis of colon length of mice; FIG. 4E: macroscopic changes in spleen size of mice; FIG. 4F: statistical analysis of spleen size of mice; and FIG. 4G: hematoxylin-eosin (HE) staining of the colon of mice;

FIGS. 5A-5E shows the results of the effect of the EcN engineered strain on oxidative stress levels and cytokines in UC mice, in which FIG. 5A: MDA content; FIG. 5B: CAT content; FIG. 5C: SOD content; FIG. 5D: IL-1B level; and FIG. 5E: IL-6 level.

### DETAILED DESCRIPTION OF THE EMBODIMENTS

The present disclosure provides an EcNc engineered strain constitutively expressing an MLH protein, where Escherichia coli Nissle 1917 is used as a chassis cell, and contains modified endogenous cryptic plasmids pMUT1 and/or pMUT2;
the modification includes inserting a gene encoding an MLH protein into the endogenous cryptic plasmids pMUT1 and/or pMUT2, where the MLH protein is a fusion protein formed by linking SOD and CAT via a flexible linker.

In one embodiment of the present disclosure, the SOD includes RtSOD with the GenBank Accession No. WP_012843426.1; the CAT includes FeCAT with the GenBank Accession No. WP_012845258.1. In an embodiment of the present disclosure, to express a fusion protein of RtSOD and CAT, the CAT gene is inserted downstream of the RtSOD gene sequence and linked via a sequence encoding a flexible linker. The fusion protein is designated MLH. The linker is a flexible peptide GGGGS (SEQ ID NO:19), which is introduced by the primer L-FeCAT-F.

The present disclosure provides a construction method for the above EcNc engineered strain, which method includes steps of: (1) inserting a gene encoding SOD into endogenous cryptic plasmids pMUT1 and/or pMUT2, and constructing linearized pMUT1-SOD and/or pMUT2-SOD plasmids by PCR method;
(2) inserting a gene encoding CAT into a prokaryotic expression vector, and constructing a linearized linker-CAT gene fragment by PCR method;
(3) ligating the linearized pMUT1-SOD and/or pMUT2-SOD plasmids of step (1) separately with the linearized linker-CAT gene fragment of step (2), to construct recombinant vectors pMUT1-MLH and/or pMUT2-MLH; and
(4) transforming the recombinant vectors pMUT1-MLH and/or pMUT2-MLH of step (3) separately into competent of Escherichia coli Nissle 1917 cells with the two endogenous cryptic plasmids pMUT1 and pMUT2 removed, to construct the EcNc engineered strain.

In one embodiment of the present disclosure, primers used in the PCR method of step (1) include pMRtSOD-X-F and pMRtSOD-X-R, where the nucleotide sequence of the pMRtSOD-X-F is set forth in SEQ ID NO: 1, and the nucleotide sequence of the pMRtSOD-X-R is set forth in SEQ ID NO: 2. In the present disclosure, when performing the PCR, the EcN recombinant endogenous cryptic plasmids pMUT1-RtSOD and/or pMUT2-RtSOD containing the RtSOD gene are used as templates, and pMUT1-RtSOD and/or pMUT2-RtSOD are linearized by reverse PCR using pMRtSOD-X-F and pMRtSOD-X-R. The construction method for the template of the present disclosure includes first extracting the pMUT1 cryptic plasmid from wild-type EcN cells, and performing linear amplification using the primers pMUT1-F/pMUT1-R; subsequently, amplifying the RtSOD-KanR fragment using primers pMUT1-RtSOD-F/pMUT1-RtSOD-R with the plasmid pGEX-4T-1-M-RtSOD as the template, where the pGEX-4T-1-M-RtSOD plasmid is obtained by inserting the RtSOD gene (WP_012843426.1) synthesized by a biotechnology company into the GTATTC site downstream of the operon of the pGEX-4T-1 plasmid, followed by operon mutation (the nucleotide sequence includes a mutation from AGCGG to GATCC). The linearized pMUT1 and RtSOD-KanR fragments are ligated by seamless cloning technology to obtain the recombinant plasmid pMUT1-RtSOD. The recombinant plasmid pMUT2-RtSOD is constructed using the same method described above, where the primers used for PCR amplification include pMUT2-F/pMUT2-R and pMUT2-RtSOD-F/pMUT2-RtSOD-R. The procedure for the reverse PCR of the present disclosure includes first pre-denaturation at 98° C. for 2 min; then denaturation at 98° C. for 10 s, annealing at 60° C. for 30 s, and extension at 72° C. for 1.5 min, for 35 cycles; and finally extension at 72° C. for 10 min.

In the present disclosure, nucleotides of CAT are codon-optimized according to the codon preference of Escherichia coli, and it is synthesized and inserted into the pET-28a (+) vector. The open reading frame (ORF) of FeCAT is genetically synthesized and inserted between the Nco I and EcoR I sites of the pET-28a (+) plasmid to generate the recombinant plasmid pET28a-FeCAT.

The PCR method of step (2) of the present disclosure includes performing PCR amplification using the recombinant plasmid pET28a-FeCAT as a template and L-FeCAT-F and L-FeCAT-R as primers, to obtain a target fragment linker-FeCAT containing the flexible linker peptide and CAT, which also carries homologous arms. The nucleotide sequence of L-FeCAT-F is set forth in SEQ ID NO: 3 and the nucleotide sequence of L-FeCAT-R is set forth in SEQ ID NO: 4.

In the present disclosure, the above linearized pMUT1-RtSOD or pMUT2-RtSOD is ligated with the linker-FeCAT fragment to obtain recombinant vectors pMUT1-MLH and pMUT2-MLH containing the FeCAT and RtSOD fusion gene.

In the embodiments of the present disclosure, two endogenous cryptic plasmids pMUT1 and pMUT2 of EcN are removed based on the principle of plasmid incompatibility to obtain EcNc cells free of cryptic plasmids. The method for removing the plasmids has been disclosed in the article (Lin Q, Jiang Z, Zhong B, Chen J Q, Lv Z B, Nie Z M. Unveiling the impact of cryptic plasmids curing on Escherichia coli Nissle 1917: massive increase in Ag43c expression. AMB Express, 2024, 14 (1): 48).

In the present disclosure, the successfully constructed plasmids pMUT1-MLH and pMUT2-MLH are separately transformed into EcNc competent cells to obtain the EcNc-pMUT1-MLH and EcNc-pMUT2-MLH engineered strains. In an embodiment of the present disclosure, the transformation is performed using the electrotransformation method. SDS-PAGE electrophoresis shows that both engineered strains constitutively express the MLH protein without IPTG induction, and it is also found that the expression level of the EcNc-pMUT2-MLH engineered strain is lower than that of the EcNc-pMUT1-MLH engineered strain.

The primer sequences used in the present disclosure are as shown in Table 1.

TABLE 1
Primers used

| | | SEQ |
|---|---|---|
| Primer | | ID |
| name | Primer sequence | NO: |
| pMRtSOD- | GAATTCCCGGGTCGACTCG | 1 |
| X-F | | |
| pMRtSOD- | CGCGGCCGCCACTTT | 2 |
| X-R | | |
| L-FeCAT- | GGCGGCGGTGGTAGCATGGAAGCGC | 3 |
| F | GCAAAGCG | |
| L-FeCAT- | TCGACCCGGGAATTCTTAGCCCGCC | 4 |
| R | ACGCTC | |
| pMUT1-F | GTTTCAGTGGTGCGTACAATTAAGG | 5 |
| pMUT1-R | GCGCTGAACGCGATTCTGAC | 6 |
| pMUT1- | GCGTTCAGCGCCCAGGCTTGACAAT | 7 |
| RtSOD-F | TAATCATCGGCTCG | |
| pMUT1- | GTACGCACCACTGAAACGAAGATCC | 8 |
| RtSOD-R | TTTGATCTTTTC | |
| pMUT2-F | GTACACACAGCGTCCCCTCTTC | 9 |
| pMUT2-R | TGGCTTAGTACAGCATCTATCGTAC | 10 |
| | ATC | |
| pMUT2- | GATGCTGTACTAAGCCACCAGGCTT | 11 |
| RtSOD-F | GACAATTAATCA | |
| pMUT2- | GGGGACGCTGTGTGTACGAAGATCC | 12 |
| RtSOD-R | TTTGATCTTTTC | |
| IL-6-F | CCCCAATTTCCAATGCTCTCC | 13 |
| IL-6-R | CGCACTAGGTTTGCCGAGTA | 14 |
| IL-1ß-F | AGGCTCCGAGATGAACAACAAA | 15 |
| IL-1B-R | GTGCCGTCTTTCATTACACAGGA | 16 |
| GAPDH-F | CCTCGTCCCGTAGACAAAATG | 17 |
| GAPDH-R | TGAGGTCAATGAAGGGGTCGT | 18 |

The present disclosure further provides the use of the above EcNc engineered strain or an EcNc engineered strain constructed using the above construction method in the preparation of an antioxidant formulation.

The EcNc-pMUT1-MLH and EcNc-pMUT2-MLH engineered strains of the present disclosure both exhibit significant SOD and CAT activities. Specifically, when the total number of colonies per ml of EcNc-pMUT1-MLH is about 7.5×10¹⁰ CFU, the SOD activity is about 600 U/mL and the CAT activity is about 15,000 U/mL, enabling the engineered strain to be used as an antioxidant formulation.

The present disclosure further provides the use of the above EcNc engineered strain or an EcNc engineered strain constructed using the above construction method in the preparation of a drug for preventing and/or treating enteritis.

In the embodiments of the present disclosure, the intragastric administration of the animal model with the EcNc engineered strain can alleviate DSS-induced UC symptoms in mice to some extent, with manifestations including mitigating weight loss, diarrhea and bloody stool symptoms, increasing the DAI score, reducing the shortening of colon length, alleviating damage to colon tissues, improving oxidative stress indicators, and decreasing the expression of inflammatory factors in colon tissues.

The EcNc engineered strain of the present disclosure can constitutively and stably express the RtSOD-FeCAT dual-enzyme fusion protein MLH without IPTG induction or antibiotic pressure. The EcNc engineered strain is rich in the highly stable SOD/CAT dual-enzyme fusion protein and has both SOD and CAT enzyme activities; the EcNc engineered strain has good protective and therapeutic effects on UC mice, with therapeutic effects stronger than those of the wild-type EcN strain, and can be used as a live biotherapeutic product (LBP) for the prevention and treatment of UC.

The present disclosure further provides a drug for preventing and/or treating enteritis, where the drug contains an active ingredient including the above EcNc engineered strain or an EcNc engineered strain constructed using the above construction method, and further comprises a pharmaceutically acceptable excipient.

For further illustration of the present disclosure, the EcNc engineered strain constitutively expressing the MLH protein of the present disclosure and the use thereof in the treatment of enteritis are described in detail below in conjunction with the examples, which, however, are not to be construed as limiting the scope of protection of the present disclosure.

Unless otherwise specified, the materials used in the examples of the present disclosure are commonly used in the art. For example, the EcN recombinant endogenous cryptic plasmids pMUT1-RtSOD and pMUT2-RtSOD containing the RtSOD gene (GenBank Accession No. WP_012843426.1) were constructed and deposited by our laboratory, which can constitutively express the RtSOD protein. Suzhou Genewiz Biotechnology Co., Ltd. was commissioned to synthesize the open reading frame (ORF) of FeCAT (GenBank Accession No. WP_012845258.1) and insert the ORF into the pET28a plasmid to generate the recombinant plasmid pET28a-FeCAT.

The two endogenous cryptic plasmids pMUT1 and pMUT2 of EcN are removed based on the principle of plasmid incompatibility to obtain EcNc cells free of cryptic plasmids. The method has been disclosed in the article (Lin Q, Jiang Z, Zhong B, Chen J Q, Lv Z B, Nie Z M. Unveiling the impact of cryptic plasmids curing on Escherichia coli Nissle 1917: massive increase in Ag43c expression. AMB Express, 2024, 14 (1): 48).

C57BL/6 mice (8 weeks old) are purchased from GemPharmatech, with a qualification certificate number: B202401030434. The mice are housed in individually ventilated cages, with six mice per cage. The mice are maintained under a standard 12-hour light/dark cycle (light period: 8:00 a.m. to 8:00 p.m.; dark period: 8:00 p.m. to 8:00 a.m.), at a controlled temperature of 26° C. and a relative humidity of 40-70%. Adequate food and water are provided. This animal experiment is approved by the Animal Ethics Committee of Zhejiang Sci-Tech University (Acceptance No.: 20231224-01).

In the examples of the present disclosure, GraphPad Prism software (version 8.0) is used for statistical analysis and graphical representation. Differences between groups are evaluated by t-test (n≥3). In statistical significance analysis, p<0.05 indicates a significant difference, p<0.01 indicates a highly significant difference, and p<0.001 indicates an extremely significant difference, which are denoted as *p<0.05, **p<0.01, and ***p<0.001, respectively. All data are presented as the mean±SEM.

### Example 1

### 1. Construction of EcNc-MLH Engineered Strain and Protein Expression

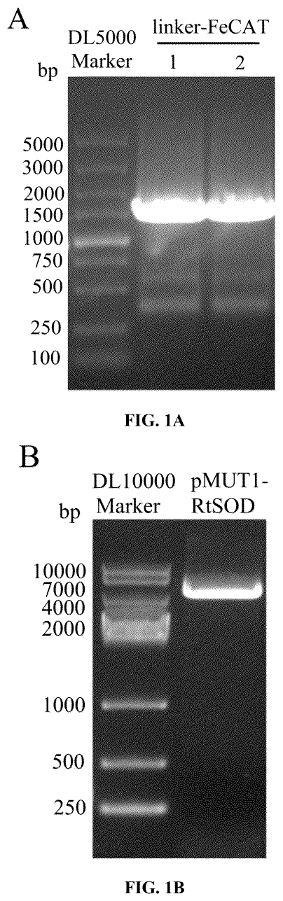

Using the plasmids pMUT1-RtSOD and pMUT2-RtSOD as templates, linearized amplification was performed by the PCR method with primers pMRtSOD-X-F and pMRtSOD-X-R to obtain linearized pMUT1-RtSOD and linearized pMUT2-RtSOD, respectively (FIG. 1B).

Using the plasmid pET28a-FeCAT as a template, a target fragment linker-FeCAT containing a flexible linker peptide and CAT was amplified with primers L-FeCAT-F and L-FeCAT-R, with homologous arms attached simultaneously (FIG. 1A).

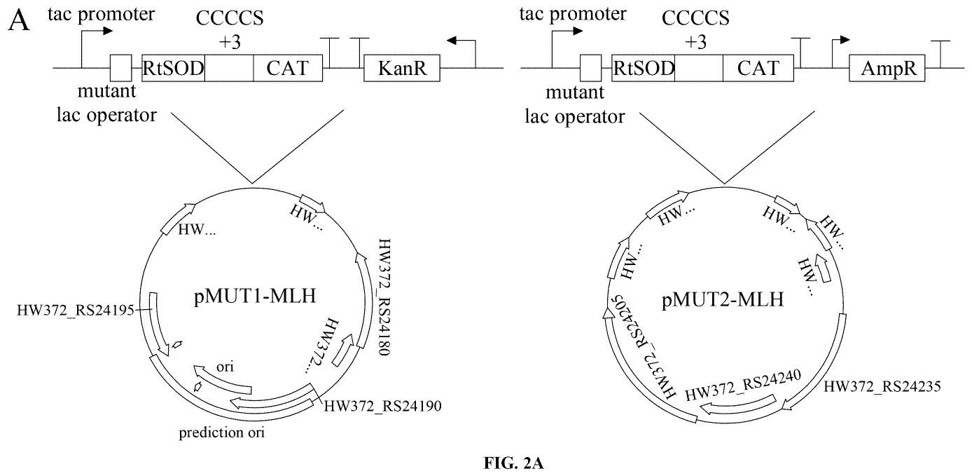

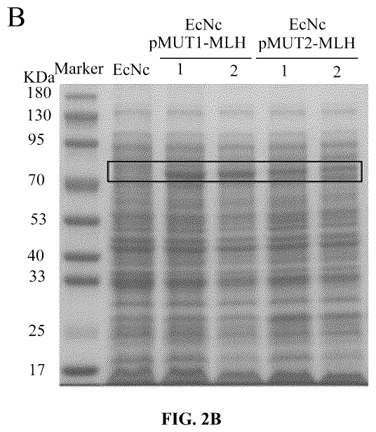

The linearized pMUT1-RtSOD and FeCAT fragments were ligated using the ClonExpress II One-Step Cloning Kit (BGI Genomics Co., Ltd., Nanjing) to construct the recombinant plasmid pMUT1-MLH. Using the same method, the linearized pMUT2-RtSOD and FeCAT fragments were ligated to construct the recombinant plasmid pMUT2-MLH (FIG. 2A).

The recombinant plasmid pMUT1-MLH and the recombinant plasmid pMUT2-MLH were separately transformed into DH5a competent cells, and their sequences were confirmed by sequencing. The strains grown under antibiotic pressure were sent to Youkang Biotechnology for sequencing, and the results were consistent with the expectations, indicating that the plasmids were successfully constructed.

The plasmids pMUT1-MLH and pMUT2-MLH constructed as described above were sequentially electrotransformed into EcNc competent cells to obtain the EcN engineered strain EcNc-MLH containing two recombinant cryptic plasmids pMUT1-MLH and pMUT2-MLH. The constitutive expression level of the MLH protein in the EcN engineered strain was analyzed by SDS-PAGE.

The results of SDS-PAGE electrophoresis are as shown in FIG. 2B, which showed that both engineered strains constitutively expressed the MLH protein without IPTG induction. It was also found that the expression level in the EcNc-pMUT2-MLH engineered strain was lower than that in the EcNc-pMUT1-MLH engineered strain. The engineered strain with high expression level, EcNc-pMUT1-MLH, was named EcNc-MLH and used for subsequent studies. The bacterial enzyme activity assay showed that when the total number of colonies per milliliter was about 7.5×10¹⁰ CFU, the SOD activity of EcNc-MLH was about 600 U/mL, and the CAT activity was about 15,000 U/mL.

### 2. Stability Identification of MLH Protein

2.1 Thermal stability of MLH: The EcNc-MLH engineered strain was cultured for 16 hours and then centrifuged at 12,000 rpm for 5 minutes. The cells were collected and subjected to ultrasonic treatment. The supernatant was collected as a crude enzyme solution of MLH. Subsequently, the crude enzyme solution was heated in a metal bath at 80° C. for 0, 20, 40, 60, 80, 100 and 120 minutes, and the SOD and CAT enzyme activities were detected separately at each time point. The SOD enzyme activity was determined using the Marklund method (national standard: GBT5009.171); the CAT enzyme activity was determined using a kit. Each group included three parallel samples. The SOD enzyme activity without heating was set as 100%, and the relative enzyme activity at each heating time point was calculated.

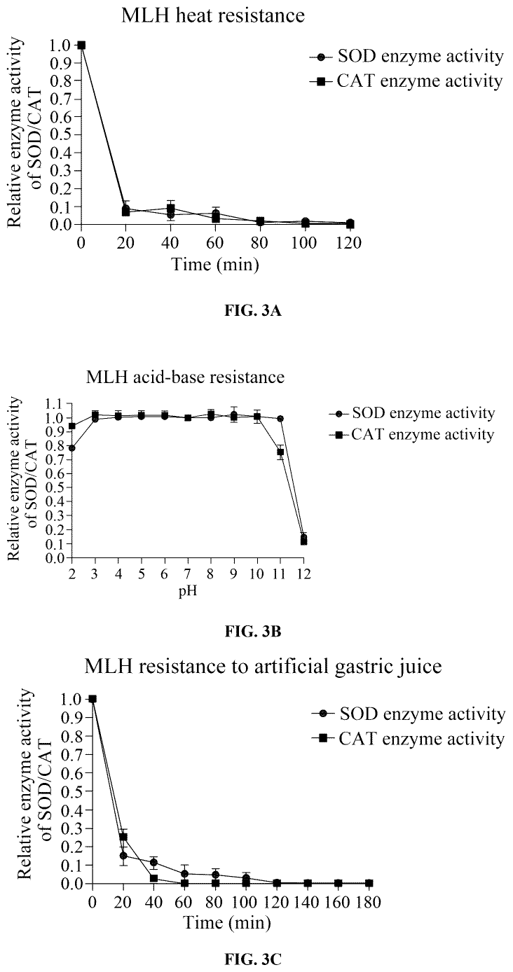

As shown in FIG. 3A, the results showed that MLH still maintained above 20% of the SOD and CAT enzyme activities after being heated at 80° C. for 20 min.

2.2 Acid-base resistance of MLH crude enzyme solution: The crude enzyme solution was diluted 10-fold with a series of buffer solutions at different pH values (pH 2-12), heated in a metal bath at 37° C. for 30 min, and then centrifuged. The supernatant was collected to determine the SOD and CAT enzyme activities. Each group included three parallel samples. The enzyme activity at pH 7 was set as 100%, and the relative SOD and CAT enzyme activities after treatment with different pH buffers were calculated.

As shown in FIG. 3B, the results showed that MLH maintained relatively stable SOD enzyme activity at pH 4-11, still maintained above 80% of SOD enzyme activity at pH 3, but exhibited a significant decrease in SOD enzyme activity at pH 2. RtSOD still maintained above 60% of SOD enzyme activity at pH 12. Meanwhile, the CAT enzyme activity of MLH maintained relatively stable at pH 2-10, the CAT enzyme activity decreased to 70% at pH 11, and the CAT enzyme activity significantly decreased at pH 12. This indicates that MLH has good resistance to high temperature and acid and base.

2.3 Resistance of MLH crude enzyme solution to artificial gastric juice/artificial intestinal juice: The crude enzyme solution was diluted 10-fold with artificial gastric juice/artificial intestinal juice, and then heated in a metal bath at 37° C. for 0 min, 20 min, 40 min, 60 min, 80 min, 100 min, 120 min, 140 min, 160 min and 180 min. The SOD and CAT enzyme activities at each time point of treatment with artificial gastric juice or artificial intestinal juice were determined. Each group included three parallel samples. The SOD and CAT enzyme activities at 0 min of treatment were set as 100%.

As shown in FIGS. 3C-3D, the results showed that the activity of MLH decreased to about 15% in 20 min, and the activity further decreased to complete loss after 2 h. Meanwhile, the CAT enzyme activity decreased sharply to about 25% in 20 min, and the activity further decreased to complete loss after 1 h (FIG. 3C). This indicates that the MLH protein has poor resistance to gastric juice; however, the live biotherapeutic product used in the present disclosure can protect MLH from destruction by gastric juice. To more accurately evaluate oral stability, an artificial intestinal fluid resistance test was conducted by simulating the human intestinal environment. The activity of MLH still remained above 70% within 3 h. The SOD activity could be maintained at 90% within the first 1.5 h. Meanwhile, the CAT enzyme activity of MLH slowly decreased to about 70% within 3 h, and could be maintained at 90% within the first 1.5 h, demonstrating excellent stability (FIG. 3D). This indicates that MLH has high stability in intestinal fluid and can retain high SOD and CAT enzyme activities after reaching the intestine via oral administration, thereby exerting a better antioxidant effect.

### 3. Evaluation of EcNc-MLH Engineered Strain in the Treatment of UC

### 3.1 Establishment of Mouse UC Model and Administration Regimen

C57BL/6 mice (8 weeks of age) were acclimatized for one week, during which all groups were provided with normal drinking water and sufficient feed, and maintained under a 12-hour light cycle. After the acclimation period, the mice were randomly grouped, with six mice in each group. A total of four groups were set: a blank control group (Control), a model group (DSS), a 10⁹ CFU/mL EcN group, and a 10⁹ CFU/mL EcNc-MLH group (SOD: 8 U/mL, CAT: 200 U/mL). The EcN group and the EcNc-MLH group were both solubilized using 1×PBS as a bacterial protectant.

Administration regimen: Three days before modeling, pre-administration began. The Control and DSS groups were subjected to intragastric administration of 200 μL of PBS on DO and D2, and the EcN group was subjected to intragastric administration of 200 μL of EcN bacterial suspension on DO and D2. At the beginning of modeling, the drinking water for the DSS group, the EcN group and the EcNc-MLH group was replaced with a 2.5% DSS solution, and the Control group continued to receive drinking water. During modeling, intragastric administration was performed once on D4, D6 and D8. At the end of modeling, the 2.5% DSS solution was replaced with drinking water. A final intragastric administration was performed on D10, and all mice were sacrificed on the evening of D15. The mice were dissected, and the colons were collected. The distal colon segments were fixed with 4% paraformaldehyde and then sent to Servicebio Biotechnology Co., Ltd. for paraffin embedding, frozen sectioning and HE staining. The remaining colon tissues were stored in a −80° C. refrigerator.

### 3.2 Disease Activity Index (DAI) Scoring

Further, the mice in the above experimental groups were subjected to DAI scoring. Throughout the experiment, the mice were weighed daily at a fixed time, with the body weight recorded, and the stool characteristics, hematochezia and activity state of the mice in all groups were observed. The DAI score is the sum of three scores for weight loss, stool characteristics and hematochezia. DAI scoring was performed on the basis of the criteria specified in Table 2.

TABLE 2
DAI scoring criteria

| Score | Weight loss (%) | Stool characteristics | Hematochezia |
|---|---|---|---|
| 0 | 0 | Normal | Normal |
| 1 | 1-5 | Slightly loose stool | Slight |
| 2 | 6-10 | Loose stool | Obvious |
| 3 | 11-15 | Diarrhea | Significant |
| 4 | >15 | | |

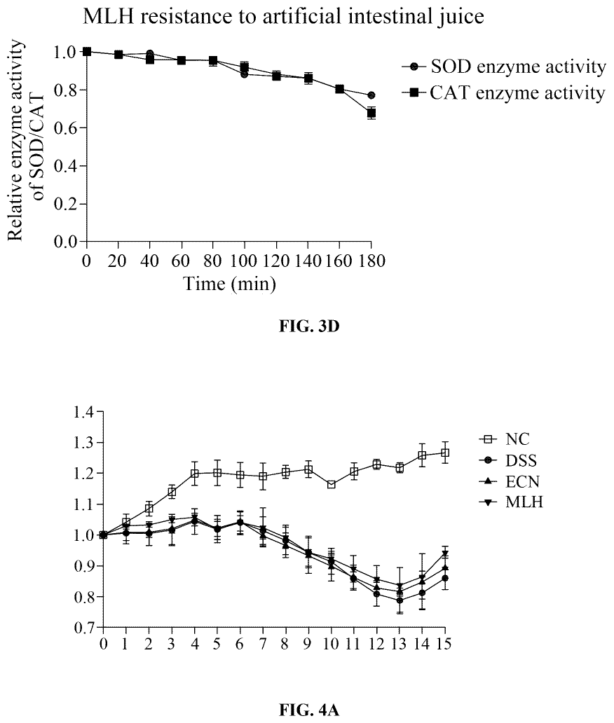

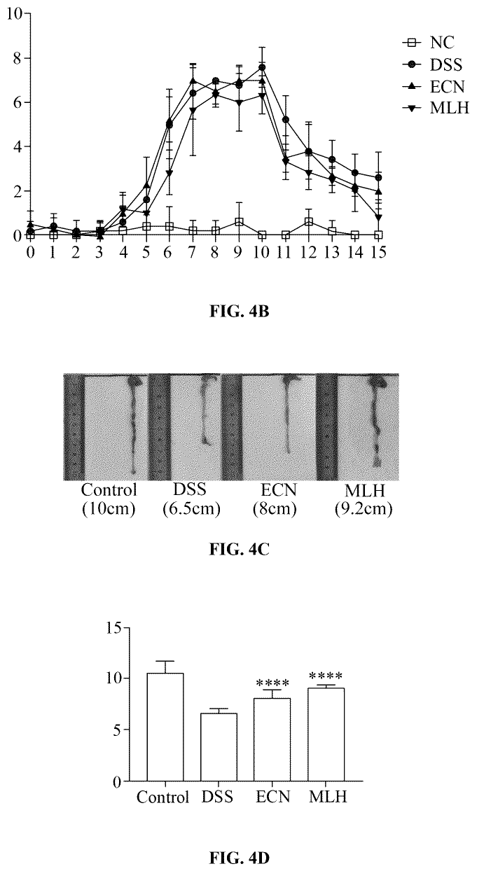

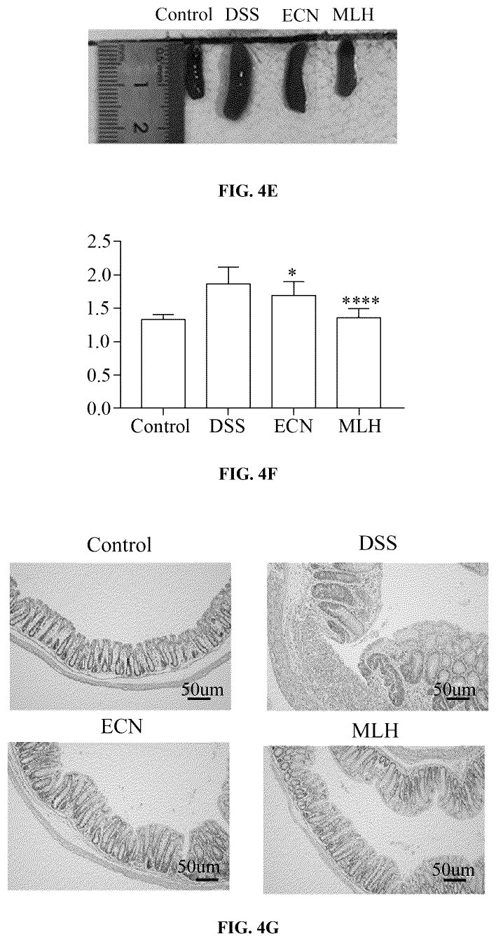

The mice were grouped for administration according to the body weight. Throughout the experiment, the mice were observed for state and weighed daily. As shown in FIG. 4A, the results showed that the overall body weight of the mice in the Control group was stable and showed a slow upward trend; in contrast, the body weight of the mice in the DSS group was stable from DO-7, and began to show a decreasing trend on D8. After stopping drinking 2.5% DSS on D10, the body weight continued to decrease and only started to recover on D14. The body weight of the ECN group began to decrease slowly on D11, and on D13 after discontinuation of administration, the weight loss was significantly less than that in the DSS group. The body weight of the MLH engineered strain group rapidly rebounded on D14. It was speculated that the MLH engineered strain has colonized the intestinal tract of the mice and plays a role.

During the experiment, the mice in the Control group exhibited glossy fur and maintained a normal activity state; in the later stage of the experiment, the mice in the DSS group exhibited dull and lusterless fur, reduced activity, and often remained curled up and stationary. The mice in the Control group had normal stool morphology and consistency, with no hematochezia observed; the mice in the DSS group had gradually loose stools starting from D6, began to develop hematochezia symptoms on D7, worsened as the experiment progressed, and eventually reached a peak on D12, with hematochezia showing a gradual remission starting from D13. Compared to the DSS group, all experimental groups showed a significant rebound in body weight and an improvement in hematochezia on D13 after discontinuation of administration. Therefore, DAI scoring was performed on the basis of the body weight changes and stool characteristics of the mice in all groups (FIG. 4B). The DAI scores of the MLH engineered strain group and the EcN group were lower than those of the DSS group. In addition, the DAI score of the MLH engineered strain group was lower than that of the EcN group. This indicates that both the MLH engineered strain and the wild-type EcN strain exhibit a certain therapeutic effect on enteritis, with the MLH engineered bacteria exhibiting a better therapeutic effect than the wild-type EcN strain.

At the end of administration, the mice in all groups were sacrificed, and the colons were collected. The colon length of the mice in all groups was measured, as shown in FIG. 4C. The mice in the Control group had a longer colon with no hyperemia or edema, and normal stool morphology remaining in the intestinal lumen; the mice in the DSS group had a shortened colon length with hyperemia and edema, and had mucoid stools in the intestinal lumen; the mice in the administration groups had a slightly longer colon than the mice in the DSS group. Among them, the wild-type EcN group and the MLH engineered strain group exhibited a significant difference in colon length from the DSS group, with the MLH engineered strain group having a longer colon than the wild-type EcN group, as shown in FIG. 4D. Moreover, the stools in the intestinal lumen of the mice in the administration groups maintained a certain morphology, and bleeding was reduced. The size of the mouse spleen is a very good indicator of systemic injury in mice. The mice in the Control group exhibited normal spleen size, whereas the mice in the DSS group exhibited a significant increase in spleen size. The spleen size of mice in the MLH group was significantly smaller than that in the DSS group. Thus, it can be concluded that the systemic injury in the mice in the MLH group was significantly less severe than that in the DSS group (FIGS. 4E-4F).

To observe the development of colon inflammation, the distal colon tissues collected as described above were observed using HE staining. The mice in the Control group exhibited an intact colonic muscular layer, with intact goblet cells in the mucosal layer, intact crypt structures and regular intestinal glands, and neatly arranged and densely packed villi with clear boundaries, and no inflammatory cell infiltration was observed; the mice in the DSS group exhibited inflammatory responses in the colon, manifested by mucosal erosion, a reduction in goblet cells, partial loss and dissolution of crypts and intestinal glands, severe submucosal edema and significant inflammatory cell infiltration; compared to the DSS group, both the wild-type EcN group and the MLH engineered strain group effectively reduced inflammatory cell infiltration, alleviated mucosal epithelial necrosis and promoted the restoration of crypt structures. Among them, the MLH engineered strain group exhibited a more significant effect (FIG. 4G).

### 3.3 Determination of MDA\SOD\CAT Oxidative Stress Indicators

SOD assay: At the end of the animal experiment, the mouse colon tissues were collected and washed with physiological saline, and an SOD sample preparation solution was added for homogenization in an ice bath, followed by centrifugation at 12,000 g for 3 min. The SOD enzyme activity was detected using the total SOD activity assay kit (WST-8 method) from Beyotime Biotechnology (Shanghai, China).

MDA assay: 0.1 g of the tissue was weighed, and 1 mL of MDA extraction solution was added for homogenization, followed by centrifugation at 8000 g for 10 min at 4° C. The supernatant was collected, and the MDA content was detected using the malondialdehyde (MDA) content assay kit (Solarbio Science & Technology Co., Ltd., Beijing, China).

CAT assay: 0.1 g of tissue, and 1 mL of CAT extraction solution was added for homogenization in an ice bath, followed by centrifugation at 8000 g for 10 min at 4° C. The supernatant was collected, and the CAT content was detected using the catalase (CAT) activity assay kit (Solarbio Science & Technology Co., Ltd., Beijing, China).

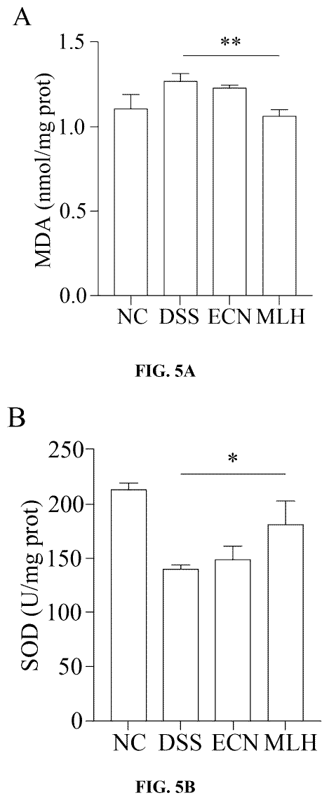

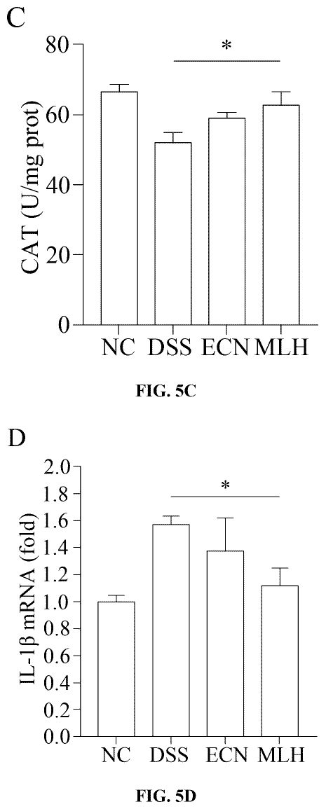

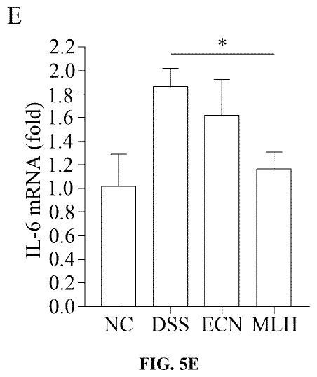

As shown in FIGS. 5A-5C, the results showed that compared to the Control group, the DSS group exhibited decreased SOD and CAT activities, along with an increased MDA content, indicating a certain degree of oxidative stress. These impairments exhibited a tendency toward alleviation after administration in all groups. In contrast, the EcNc-MLH group exhibited significant differences in both SOD and CAT activities from the DSS group. The results suggest that EcNc-MLH has a certain antioxidant effect.

### 3.4 Determination of Inflammatory Factors

1 mL of RNA extraction reagent and three 3 mm grinding beads were added to a pre-cooled grinding tube. The colon tissue (5-20 mg) was added to the tube and homogenized using a tissue grinder until no visible debris remained. Then, centrifugation was performed at 12,000 rpm for 10 minutes at 4° C., and the supernatant was collected. Total RNA was extracted using the Direct-zol RNA Microprep kit (Jianshi Biotechnology, Beijing, China), and reverse transcription was performed with the Evo M-MLV Plus cDNA synthesis kit (AG Bio, Hunan, China) to obtain cDNA. Primers for the target genes IL-6 and IL-1B and the reference gene GAPDH were designed using the qPCR primer design tool from NCBI. The primer sequences are shown in Table 1. The primers were diluted to 10 μM with double-distilled water before use, and the qPCR experiment was performed according to the instructions of the qPCR Master Mix enzyme.

As shown in FIGS. 5D-5E, the results showed that compared to the Control group, the DSS group exhibited a significant increase in the gene expression levels of IL-6/IL-1β in mouse colon tissues. However, compared to the DSS group, the mRNA levels of IL-6/IL-1β decreased after administration, and the EcNc-MLH group exhibited a significant difference from the DSS group. Thus, it can be concluded that the EcNc-MLH engineered strain can reduce the DSS-induced inflammatory response in colon tissues, thereby alleviating the damage to colon tissues.

Although the examples described above have provided a detailed description of the present disclosure, they are only some, rather than all, of the examples of the present disclosure. All other examples that can be obtained according to the examples of the present disclosure without involving any inventive effort shall fall within the scope of protection of the present disclosure.

What is claimed is:

1. An E. coli Nissle 1917 strain cured of the cryptic plasmid (EcNc) engineered strain constitutively expressing an MnSOD-Linker-Catalase (MLH) protein, wherein Escherichia coli Nissle 1917 is used as a chassis cell and comprises modified endogenous cryptic plasmids pMUT1 and/or pMUT2;
the modification comprises inserting a gene encoding an MLH protein into the endogenous cryptic plasmids pMUT1 and/or pMUT2, wherein the MLH protein is a fusion protein formed by linking Superoxide dismutase (SOD) and Catalase (CAT) via a flexible linker.

2. The EcNc engineered strain according to claim 1, wherein the SOD comprises RtSOD with the GenBank Accession No. WP_012843426.1; The CAT comprises FeCAT with the GenBank Accession No. WP_012845258.1.

3. A method for constructing the EcNc engineered strain according to claim 1, comprising steps of: (1) inserting a gene encoding SOD into endogenous cryptic plasmids pMUT1 and/or pMUT2, and constructing linearized pMUT1-SOD and/or pMUT2-SOD plasmids by a PCR method;
(2) inserting a gene encoding CAT into a prokaryotic expression vector, and constructing a linearized linker-CAT gene fragment by a PCR method;
(3) ligating the linearized pMUT1-SOD and/or pMUT2-SOD plasmids of step (1) separately with the linearized linker-CAT gene fragment of step (2), to construct recombinant vectors pMUT1-MLH and/or pMUT2-MLH; and
(4) transforming the recombinant vectors pMUT1-MLH and/or pMUT2-MLH of step (3) separately into Escherichia coli Nissle 1917 with the two endogenous cryptic plasmids pMUT1 and pMUT2 removed, to construct the EcNc engineered strain.

4. The method according to claim 3, wherein the SOD comprises RtSOD with the GenBank Accession No. WP_012843426.1; The CAT comprises FeCAT with the GenBank Accession No. WP_012845258.1.

5. The method according to claim 3, wherein primers used in the PCR method of step (1) comprise pMRtSOD-X-F and pMRtSOD-X-R, wherein the nucleotide sequence of the pMRtSOD-X-F is set forth in SEQ ID NO: 1, and the nucleotide sequence of the pMRtSOD-X-R is set forth in SEQ ID NO: 2.

6. The method according to claim 3, wherein primers used in the PCR method of step (2) comprise L-FeCAT-F and L-FeCAT-R, wherein the nucleotide sequence of the L-FeCAT-F is set forth in SEQ ID NO: 3, and the nucleotide sequence of the L-FeCAT-R is set forth in SEQ ID NO: 4.

7. An antioxidant formulation, comprising the EcNc engineered strain according to claim 1.

8. An antioxidant formulation, comprising EcNc engineered strain constructed by the method according to claim 3.

9. A method for preventing and/or treating enteritis, comprising administrating the EcNc engineered strain according to claim 1 to a subject in need.

10. The method according to claim 9, wherein types of the enteritis comprise ulcerative colitis.

11. A drug for preventing and/or treating enteritis, wherein the drug comprises an active ingredient comprising the EcNc engineered strain according to claim 1, and further comprises a pharmaceutically acceptable excipient.

12. The drug according to claim 11, wherein types of the enteritis comprise ulcerative colitis.

13. The method according to claim 11, wherein types of the drug for preventing and/or treating enteritis comprise a live biotherapeutic product.

14. A drug for preventing and/or treating enteritis, wherein the drug comprises an active ingredient comprising an EcNc engineered strain constructed by the method according to claim 3, and further comprises a pharmaceutically acceptable excipient.

15. The drug according to claim 14, wherein types of the enteritis comprise ulcerative colitis.

16. The method according to claim 14, wherein types of the drug for preventing and/or treating enteritis comprise a live biotherapeutic product.


SEQUENCE LISTING (19 sequences)

SEQ ID NO: 1 - DNA, 19 bp, synthetic construct
  source 1..19: mol_type = other DNA; note = Primer pMRtSOD-X-F
gaattcccgggtcgactcg

SEQ ID NO: 2 - DNA, 15 bp, synthetic construct
  source 1..15: mol_type = other DNA; note = Primer pMRtSOD-X-R
cgcggccgccacttt

SEQ ID NO: 3 - DNA, 33 bp, synthetic construct
  source 1..33: mol_type = other DNA; note = Primer L-FeCAT-F
ggcggcggtggtagcatggaagcgcgcaaagcg

SEQ ID NO: 4 - DNA, 31 bp, synthetic construct
  source 1..31: mol_type = other DNA; note = Primer L-FeCAT-R
tcgacccgggaattcttagcccgccacgctc

SEQ ID NO: 5 - DNA, 25 bp, synthetic construct
  source 1..25: mol_type = other DNA; note = Primer pMUT1-F
gtttcagtggtgcgtacaattaagg

SEQ ID NO: 6 - DNA, 20 bp, synthetic construct
  source 1..20: mol_type = other DNA; note = Primer pMUT1-R
gcgctgaacgcgattctgac

SEQ ID NO: 7 - DNA, 39 bp, synthetic construct
  source 1..39: mol_type = other DNA; note = Primer pMUT1-RtSOD-F
gcgttcagcgcccaggcttgacaattaatcatcggctcg

SEQ ID NO: 8 - DNA, 37 bp, synthetic construct
  source 1..37: mol_type = other DNA; note = Primer pMUT1-RtSOD-R
gtacgcaccactgaaacgaagatcctttgatcttttc

SEQ ID NO: 9 - DNA, 22 bp, synthetic construct
  source 1..22: mol_type = other DNA; note = Primer pMUT2-F
gtacacacagcgtcccctcttc

SEQ ID NO: 10 - DNA, 28 bp, synthetic construct
  source 1..28: mol_type = other DNA; note = pMUT2-R
tggcttagtacagcatctatcgtacatc

SEQ ID NO: 11 - DNA, 37 bp, synthetic construct
  source 1..37: mol_type = other DNA; note = Primer pMUT2-RtSOD-F
gatgctgtactaagccaccaggcttgacaattaatca

SEQ ID NO: 12 - DNA, 37 bp, synthetic construct
  source 1..37: mol_type = other DNA; note = Primer pMUT2-RtSOD-R
ggggacgctgtgtgtacgaagatcctttgatcttttc

SEQ ID NO: 13 - DNA, 21 bp, synthetic construct
  source 1..21: mol_type = other DNA; note = Primer IL-6-F
ccccaatttccaatgctctcc

SEQ ID NO: 14 - DNA, 20 bp, synthetic construct
  source 1..20: mol_type = other DNA; note = Primer IL-6-R
cgcactaggtttgccgagta

SEQ ID NO: 15 - DNA, 22 bp, synthetic construct
  source 1..22: mol_type = other DNA; note = Primer IL-1 beta-F
aggctccgagatgaacaacaaa

SEQ ID NO: 16 - DNA, 23 bp, synthetic construct
  source 1..23: mol_type = other DNA; note = Primer IL-1 beta-R
gtgccgtctttcattacacagga

SEQ ID NO: 17 - DNA, 21 bp, synthetic construct
  source 1..21: mol_type = other DNA; note = Primer GAPDH-F
cctcgtcccgtagacaaaatg

SEQ ID NO: 18 - DNA, 21 bp, synthetic construct
  source 1..21: mol_type = other DNA; note = Primer GAPDH-R
tgaggtcaatgaaggggtcgt

SEQ ID NO: 19 - protein, 5 aa, synthetic construct
  source 1..5: note = A flexible peptide
GGGGS